(19) United States
(12) Patent Application Publication
(10) Pub. No.: US 2019/0242963 A1
(43) Pub. Date: Aug. 8, 2019

(54) IMPROVEMENT OF SIMULTANEOUS MEASURE OF THE TEMPERATURE AND THE DISPLACEMENT MEASURED WITH MAGNETIC RESONANCE ACOUSTIC RADIATION FORCE IMAGING

(71) Applicant: KONINKLIJKE PHILIPS N.V., EINDHOVEN (NL)

(72) Inventor: CHARLES MOUGENOT, Ontario (CA)

(21) Appl. No.: 16/337,458

(22) PCT Filed: Sep. 25, 2017
(86) PCT No.: PCT/EP2017/074153

§ 371 Date: Mar. 28, 2019

Related U.S. Application Data
(60) Provisional application No. 62/403,812, filed on Oct. 4, 2016.

(51) Int. Cl. G01R 33/48; A61N 7/02; G01R 33/563

(52) U.S. Cl. CPC G01R 33/4804; A61N 7/02; A61N 2007/0078; G01R 33/4814; G01R 33/56358

(57) ABSTRACT

In Magnetic Resonance Acoustic Radiation Force Imaging (MR-ARFI), an MR imaging device (10) performs gradient echo imaging including successive MR dynamics with opposite encoding of displacement to generate MR-ARFI data of a subject comprising successive image frames with opposite displacement encoding. An ultrasound device (12) applies sonication to the subject during the gradient echo imaging. An electronic processor (22) performs MR-ARFI data processing applied to image elements at image frames of the MR-ARFI data. A displacement is computed (30) for the image element at the image frame as proportional to a phase difference between the image element in the image frame and the image element in a succeeding or preceding image frame with opposite displacement encoding. The computed displacement is corrected (32) for a temperature change between the image frame and the succeeding or preceding image frame. The temperature change is determined using the MR-ARFI data.

### FIELD

The following relates generally to the medical ultrasound arts, medical imaging arts, acoustic radiation force imaging arts, and related arts.

### BACKGROUND

Magnetic Resonance Acoustic Radiation Force Imaging (MR-ARFI) is used to image the acoustic radiation force generated in tissue during a medical ultrasound examination or a medical ultrasound therapeutic procedure. In MR-ARFI, the ultrasound pulses to be imaged are applied during concurrent application by a magnetic resonance (MR) imaging device of motion encoding magnetic field gradients in order to monitor the displacement induced by these ultrasound pulses. This displacement is proportional to the local acoustic intensity, and provides a real-time imaging measurement of the therapeutic beam shape. By way of non-limiting illustration, MR-ARFI finds application in various therapeutic ultrasound procedures such as High intensity focused ultrasound (HIFU) medical procedures. For example, MR-ARFI imaging may be used to visualize the focal point during HIFU test pulses, or to assess refocusing of the HIFU beam prior to MR-HIFU treatments.

MR-ARFI sequences have been designed of the gradient-echo (GRE) and spin-echo (SE) sequence types. For each of these sequences these displacements are encoded by motion encoding gradients as phase variations. To separate the phase variation due to the displacement from other sources of phase variations, such as magnetic field homogeneities and/or temperature, a known approach is to apply two successive MR dynamics (or image frames) labelled n and n−1, with opposite encoding of the displacement used in the two image frames. Different methods exist to generate this opposite encoding of the displacement. One known approach entails inversing the polarity of the motion encoding gradient every dynamic. As result the difference of phase φₙ−φₙ₋₁ between two successive dynamics are proportional to the displacement Dₙ according to:

$${D}_{n} = \frac{{ϕ}_{n} - {ϕ}_{n - 1}}{4 π \cdot γ \cdot {B}_{0} \cdot {G}_{A} \cdot {G}_{D} \cdot {S}_{n}} \qquad ( 1 )$$

In Equation (1), γ represents the gyromagnetic ratio (42.58 MHz/T), B₀ represents the magnetic field strength (e.g., 1 Tesla in a non-limiting illustrative example), G_{A} represents the amplitude of the motion encoding gradient (e.g. 1 ms in a non-limiting illustrative example), G_{D} represents the duration of the motion encoding gradient (e.g., 30 mT/m in a non-limiting illustrative example), and Sₙ represents the polarity of the encoding (Sₙ=1 for odd dynamics n=2 k+1, and Sₙ=−1 for even dynamics n=2 k). If the absolute displacement amplitude is not of interest (e.g., when using MR-ARFI imaging to visualize the spatial position of the focused HIFU beam), Equation (1) can be written as the proportionality expression:

$${D}_{n} ∝ \frac{{ϕ}_{n} - {ϕ}_{n - 1}}{{S}_{n}} \qquad ( 1 a )$$

GRE sequence implementations of MR-ARFI offer an additional advantage, namely providing simultaneous monitoring of the temperature. This is of particular values since the ultrasound pulses can produce localized tissue heating. The proton resonance frequency equation states that the temperature increase Tₙ is proportional the phase variation as set forth below:

$${T}_{n} = \frac{{ϕ}_{n} - {ϕ}_{0}}{2 π \cdot γ \cdot α \cdot {B}_{0} \cdot {T}_{E}} \qquad ( 2 )$$

In Equation (2), α corresponds to the chemical shift (e.g. 0.0094 ppm/° C. in a non-limiting example), and T_{E} represents the echo time (i.e. time-to-echo) of the GRE sequence, equal to 30 ms in some non-limiting examples.

The following discloses a new and improved systems and methods.

### SUMMARY

In one disclosed aspect, a Magnetic Resonance Acoustic Radiation Force Imaging (MR-ARFI) apparatus is disclosed. A magnetic resonance (MR) imaging device is configured to perform gradient echo (GRE) imaging including successive MR dynamics with opposite encoding of displacement to generate MR ARFI data of a subject in which the MR-AFRI data comprises successive image frames with opposite encoding of displacement. An ultrasound device is configured to apply sonication to the subject over sonication time intervals during the GRE imaging. An electronic processor is programmed to perform an MR-ARFI data processing method applied to image elements at image frames of the MR-AFRI data, including: computing a displacement for the image element at the image frame as proportional to a difference between the phase of the image element in the image frame and the phase of the image element in a succeeding or preceding image frame with opposite encoding of displacement; and correcting the computed displacement for a temperature change for the image element between the image frame and the succeeding or preceding image frame to generate a temperature-corrected displacement for the image element at the image frame, wherein the temperature change is determined using the MR-AFRI data.

In another disclosed aspect, a non-transitory storage medium stores instructions readable and executable by an electronic processor to perform a Magnetic Resonance Acoustic Radiation Force Imaging (MR-ARFI) method operating on MR-AFRI data of a subject comprising successive image frames with opposite encoding of displacement acquired during sonication of the subject over sonication time intervals. The MR-ARFI method comprises computing a temperature-corrected displacement for an image element at an image frame of the MR-AFRI data from the phase of the image element at the image frame and the phase of the image element at a succeeding or preceding image frame with opposite encoding of displacement. The computing is repeated for at least one of: (1) all image elements of the image frame to generate a temperature-corrected displacement image; and (2) a contiguous plurality of image frames of the MR-AFRI data to generate a temperature-corrected displacement versus time profile for the image element.

In another disclosed aspect, a Magnetic Resonance Acoustic Radiation Force Imaging (MR-ARFI) method comprises performing gradient echo (GRE) imaging using a magnetic resonance (MR) imaging device to acquire MR ARFI data of a subject in which the MR-AFRI data comprises successive image frames with opposite encoding of displacement, and applying sonication to the subject over sonication time intervals during the GRE imaging using an ultrasound device. Using an electronic processor, for image elements at image frames of the MR ARFI data: (i) displacement is computed as proportional to a difference between the phase in the image frame and the phase in a succeeding or preceding image frame with opposite encoding of displacement, and (ii) the computed displacements are corrected for a temperature change between the image frame and the succeeding or preceding image frame wherein the temperature change is determined using the MR AFRI data.

One advantage resides in providing more precise displacement measurement by Magnetic Resonance Acoustic Radiation Force Imaging (MR-ARFI).

Another advantage resides in providing more accurate displacement measurement by MR-ARFI.

Another advantage resides in providing displacement measurement by MR-ARFI with reduced oscillation artifacts.

Another advantage resides in providing displacement measurement by MR-ARFI with reduced artifacts.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates an MR-ARFI device.

FIGS. 2-15 present MR-ARFI data as described herein.

### DETAILED DESCRIPTION

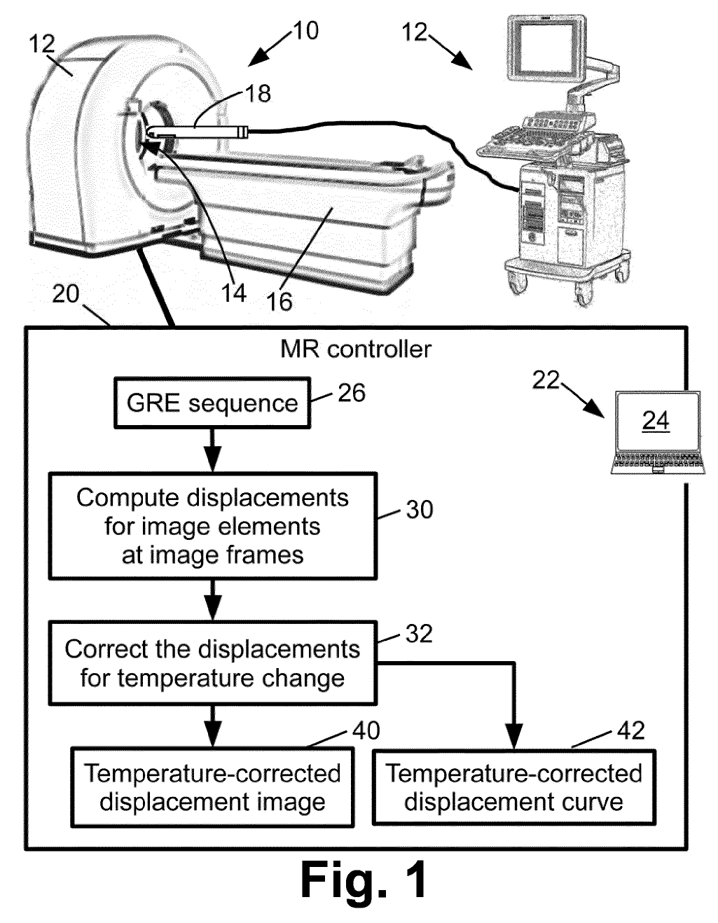

With reference to FIG. 1, Magnetic Resonance Acoustic Radiation Force Imaging (MR-ARFI) is performed by a magnetic resonance (MR) imaging device 10 in conjunction with an ultrasound device 12. The MR imaging device 10 includes a housing 12 defining a bore or other examination region 14 into which a medical patient or other subject is placed for MR imaging, e.g. using an illustrative patient couch 16. The MR imaging device 10 includes various components not shown in FIG. 1, such as an MR magnet operating to generate a static (B₀) magnetic field in the examination region 14, magnetic field gradient coils to superimpose magnetic field gradients on the Bo field, and one or more radio frequency (RF) coils and/or coil arrays operating to excite and detect magnetic resonance in the subject. The MR imaging device 10 may, for example, comprise an Ingenia™ 1.5 Tesla or 3.0 Tesla imaging system available from Koninklijke Philips N.V. (Eindhoven, the Netherlands). The ultrasound device 12 includes, or connects with, an ultrasound probe 18 and operates to drive an ultrasound transducer or transducer array (not shown) of the ultrasound probe 18 to apply sonication to the subject over sonication time intervals. Each sonication may, for example, employ a radio frequency (e.g. MHz) ultrasonic pulse burst.

The MR imaging device 10 is controlled by an MR controller 20 comprising an electronic processor and non-transitory storage media, for example embodied by an illustrative computer 22 and/or by one or more dedicated MR control electronic processors and/or dedicated non-transitory storage media (not shown). The MR controller operates the MR imaging device 10 to execute chosen MR sequences for exciting, spatially encoding, and reading out MR data. The MR controller 20 includes at least one display 24 for displaying MR images or other visualization of MR data acquired by the MR imaging device 10. The MR controller 20 also includes one or more non-transitory storage media (not shown) which may, by way of non-limiting illustration, comprise a hard disk or other magnetic storage medium, an optical disk or other optical storage medium, a flash memory, solid state drive (SSD) or other electronic storage medium, various combinations thereof, or so forth.

For the subject MR-ARFI application, the MR controller 20 stores a gradient echo (GRE) pulse sequence 26 used for MR-ARFI data acquisition. In the GRE pulse sequence 26, displacements are encoded by motion encoding gradients as phase variations. The MR imaging device 10 performs gradient echo (GRE) imaging by executing the GRE pulse sequence 26. The GRE imaging includes successive MR dynamics with opposite encoding of displacement to generate MR-ARFI data of a subject loaded into the examination region 14. The acquired MR-AFRI data comprises successive image frames with opposite encoding of displacement produced by respective successive MR dynamics with opposite encoding of displacement. Concurrently, the ultrasound device 12 is connected with the ultrasound probe 18 disposed with the subject in the examination region 14 to apply sonication to the subject over sonication time intervals during the GRE imaging. The sonications produce displacement in tissue of the subject.

In an operation 30, displacements are computed for image elements at image frames of the GRE imaging. This may be done, for example, using Equation (1) presented previously herein, or using Equation (1a) if absolute displacement amplitude is not of interest. The image elements may be image pixels in the case of two-dimensional (2D) MR data acquisition, or may be image voxels in the case of three-dimensional (3D) MR data acquisition. In general, for an image element at an image frame, the displacement for the image element at the image frame is computed as proportional to a difference between the phase of the image element in the image frame and the phase of the image element in a succeeding or preceding image frame with opposite encoding of displacement. In the illustrative examples herein, the displacement for the image element at the image frame (denoted herein as n) is computed as proportional to the difference between the phase φₙ of the image element in the image frame n and the phase φₙ₋₁ of the image element in the preceding image frame n−1 with opposite encoding of displacement.

Optionally, the temperature of the image element at the image frame may also be computed from the MR-ARFI data, e.g. using Equation (2). The expression of Equation (2) actually yields a temperature difference between a reference image frame denoted n=0 which has phase denoted φ₀, and the frame n. In some embodiments disclosed herein (particularly in the phase plots), φ₀ is designated as zero degrees (i.e. φ_{c}=0) for simplicity, although this is not required.

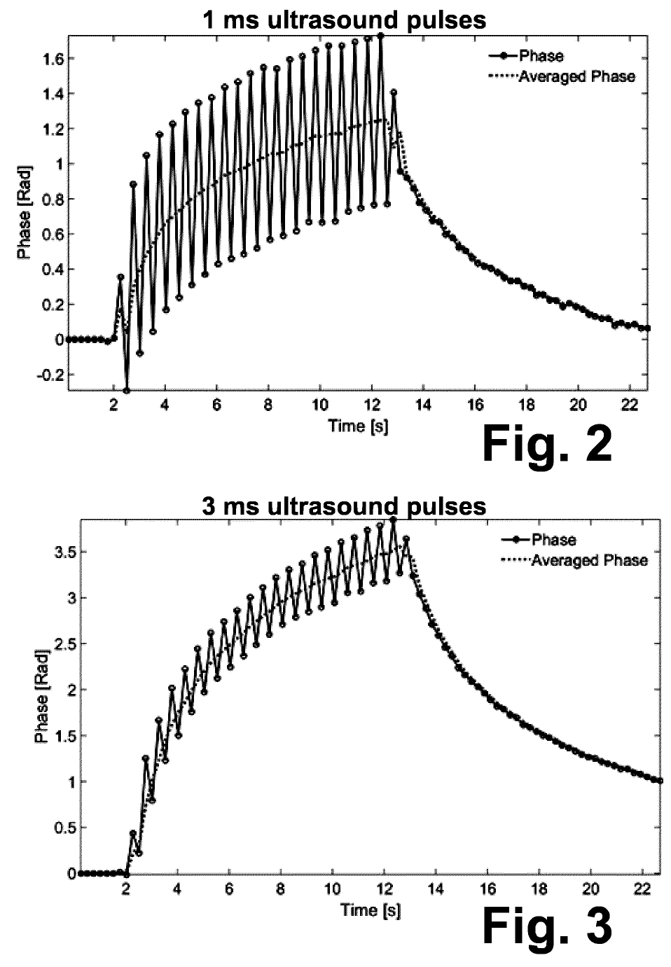

With brief reference now to FIGS. 2 and 3, considering Equations (1) (or (1a)) and (2), it is recognized herein that both the displacement Dₙ and the temperature variation Tₙ are encoded as function of time in the same phase maps φₙ. FIGS. 2 and 3 present phase variation (φₙ−φ₀) as a function of time for two examples of sonication at 200 Wac. The example of FIG. 2 used ultrasound pulses of 1 ms inducing small heating of 5° C.; whereas, the example of FIG. 3 used ultrasound pulses of 3 ms inducing a larger heating of 15° C. Both sonication examples have duration of 11 seconds, i.e. were applied between 2 sec and 13 sec using the time reference of the abscissa of FIGS. 2 and 3.

As seen in FIGS. 2 and 3, when ultrasound pulses are applied over the sonication time interval between time 2 sec and 13 sec, the phase oscillates due to the presence of displacement encoded with a different polarity at each dynamic. In a known approach for extracting both displacement and temperature from the phase maps, Equation (1) is used to process the displacement Dₙ, and the average of the phase over two successive dynamics is used in conjunction with Equation (2) to obtain the average temperature Tₙ^{A} as follows:

$${T}_{n}^{A} = \frac{\frac{1}{2} ( {ϕ}_{n} + {ϕ}_{n - 1} ) - {ϕ}_{0}}{2 π \cdot γ \cdot α \cdot {B}_{0} \cdot {T}_{E}} = \frac{{T}_{n} + {T}_{n - 1}}{2} \qquad ( 3 )$$

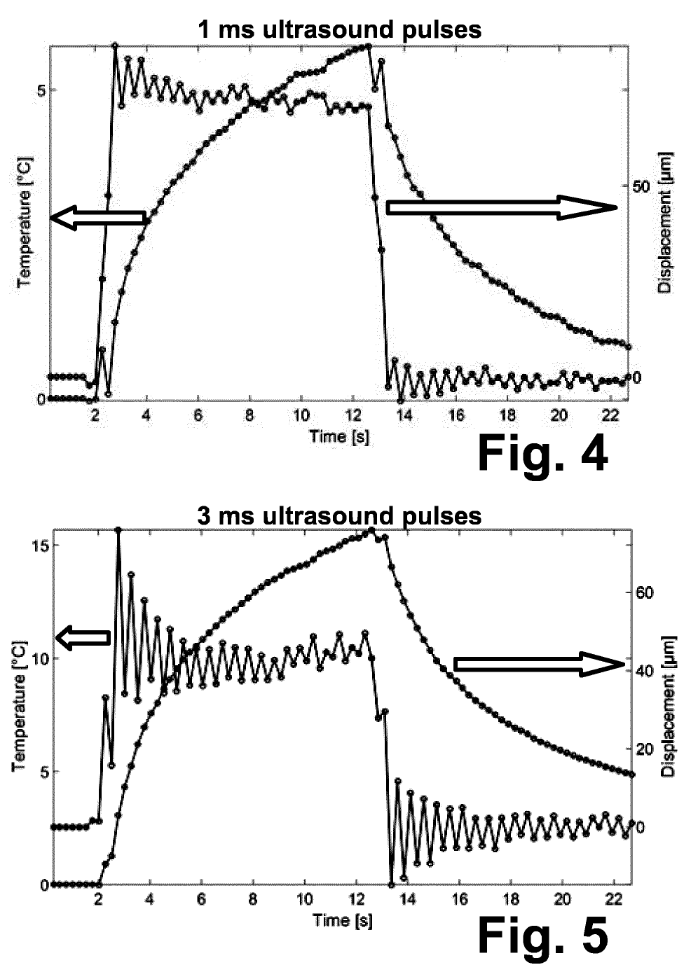

With reference now to FIGS. 4 and 5, the resulting average temperature Tₙ^{A} and displacement Dₙ are shown, with Tₙ^{A} plotted against the left ordinate and Dₙ plotted against the right ordinate. FIG. 4 plots average temperature Tₙ^{A} and displacement Dₙ for the data of FIG. 2. FIG. 5 plots average temperature Tₙ^{A} and displacement Dₙ for the data of FIG. 3. The results presented in FIGS. 4 and 5 demonstrate that this conventional approach provides a good estimation of the temperature change over the sonication time interval; however the estimation of the displacement exhibits substantial oscillations. As recognized herein, the root cause of these displacement oscillations is that the displacement is measured based on Equation (1) which assumes that the phase change (φₙ−φₙ₋₁) is solely due to change in displacement, and has no temperature change component. Said another way, Equation (1) assumes that the temperature is same at dynamics n and n−1. As recognized herein, this assumption is not reasonable in many practical therapeutic (medical) ultrasound treatments, such as in typical High Intensity Focused Ultrasound (HIFU) medical procedures. Typically, a high imaging frame rate is used in an effort to ensure that the temperature change between successive image frames is negligibly small. However, the data of FIGS. 2-5 employed a high imaging frame rate (i.e. short imaging frame period, namely 252 ms per dynamic in FIGS. 2-5). Nevertheless, significant displacement oscillations are observed in FIGS. 4 and 5. The assumption of negligible temperature change between successive dynamics n−1 to n is recognized herein to be unreasonable, especially in presence of a rapid temperature increase, e.g. 1° C./s, which is frequently produced during ultrasonic thermo-therapy.

In view of the foregoing, the MR-ARFI data processing method of FIG. 1 includes the operation 30 of computing a displacement for the image element at the image frame as proportional to a difference (φₙ−φₙ₋₁) between the phase of the image element in the image frame n and the phase of the image element in the preceding image frame (n−1) with opposite encoding of displacement (or, alternatively, a difference φₙ₊₁−φₙ between the phase of the image element in the image frame n and the phase of the image element in to succeeding image frame n+1 with opposite encoding of displacement). The disclosed MR-ARFI data processing method of FIG. 1 further includes an operation 32 in which the displacement computed in operation 30 is corrected for a temperature change for the image element between the image frame and the preceding image frame to generate a temperature-corrected displacement for the image element at the image frame. The temperature change is determined using the MR-AFRI data, e.g. using Equation (2) or variants thereof as disclosed in various embodiments herein.

The foregoing MR-ARFI data processing 30, 32 may be applied to a particular image element (e.g. pixel or voxel) and a particular image frame n. This processing 30, 32 may be repeated for all image elements of the image frame n to generate a temperature-corrected displacement image 40 for the image frame n. Such an image may be useful, for example, for visualizing the focal point during HIFU test pulses.

Additionally, or alternatively, the processing 30, 32 may be repeated for a contiguous plurality of image frames of the MR-AFRI data to generate a temperature-corrected displacement-versus-time profile or curve 42 for the image element. Such a curve may be useful, for example, to assess refocusing of the HIFU beam prior to MR-HIFU treatments (in this case, the image element is preferably chosen to be at the beam focus).

The displacement provided by operation 30 is thus improved in operation 32 to obtain a more accurate quantification of the displacement. In some embodiments disclosed herein, to improve the displacement Dₙ for the image element at image frame n, the operation 32 obtains an estimation of the temperature variation occurring between dynamics n and n−1. This estimation can be obtained from the average temperature as an example. The phase variation associated with this estimated temperature variation between dynamics n and n−1 is then subtracted from the phase difference (φₙ−φₙ₋₁) used in Equation (1).

In the following, some more detailed illustrative embodiments are described.

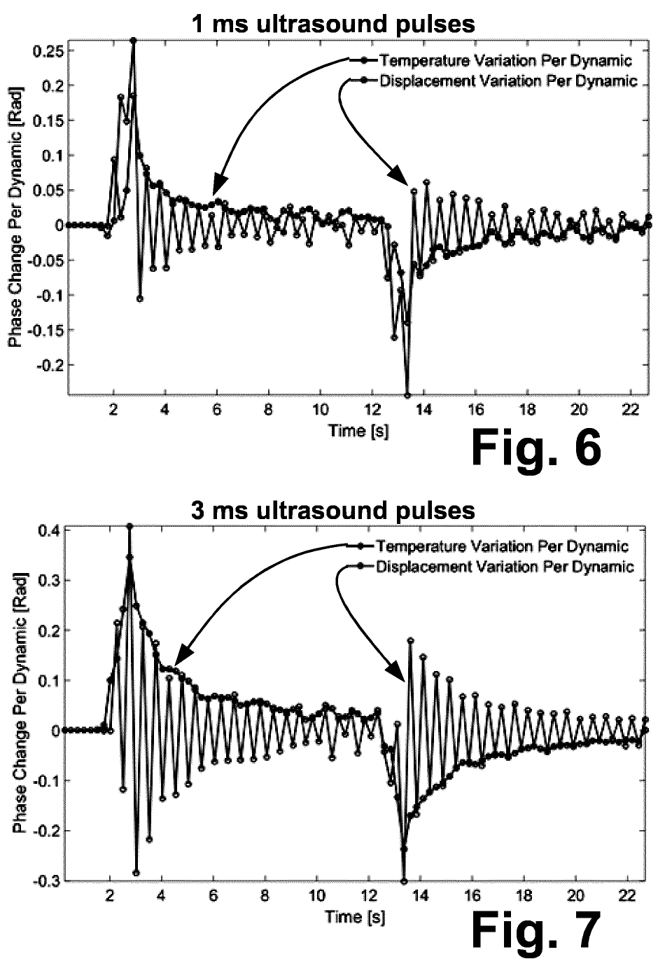

With reference to FIGS. 6 and 7, the variation of temperature can be estimated as a numerically estimated temperature derivative as follows:

$$\frac{1}{2} ( {T}_{n + 1}^{A} - {T}_{n - 1}^{A} ) \qquad ( 3 D )$$

and the variation of displacement (Dₙ−Dₙ₋₁) is plotted in FIGS. 6 and 7 for the sonications shown in FIGS. 2 and 3, respectively. More particularly, to compare these two quantities, both were converted to a phase change per dynamic using the conversion factors displacement-to-phase of Equation (1) and temperature-to-phase of Equation (2), respectively, and then plotted in FIGS. 6 and 7. Except for the sign of gradient not used for the conversion displacement to phase, the strong correlation of those two quantities confirms that the phase change associated to the temperature variation per dynamic can be used to correct the apparent noise in the estimation of the displacement.

A temperature-corrected displacement Dₙ^{C} can then be computed using this numerically estimated temperature derivative as follows:

$${D}_{n}^{C} = {D}_{n} - \frac{α \cdot {T}_{E}}{2 \cdot {G}_{A} \cdot {G}_{D} \cdot {S}_{n}} \cdot \frac{{T}_{n + 1}^{A} - {T}_{n - 1}^{A}}{2} \qquad ( 4 )$$

In the approach of Equation (4), operation 32 is performed as follows. The temperature change is estimated by numerically estimating a temperature derivative

$$\frac{{T}_{n + 1}^{A} - {T}_{n - 1}^{A}}{2}$$

for the image element at the image frame n from the MR-AFRI data (Equation (3D)), and the displacement computed in operation 30 is corrected using the temperature change as per Equation (4) to generate the temperature-corrected displacement for the image element at the image frame.

In an alternative formulation, based on Equations (1) and (3) the temperature-corrected displacement Dₙ^{C} can also be expressed as function of the phase φₙ or the weighted average of the displacements Dₙ₊₁, Dₙ, and Dₙ₋₁ computed in operation 30 so as to express the temperature-corrected displacement Dₙ^{C} as:

$${D}_{n}^{C} = \frac{- {ϕ}_{n + 1} + 3 {ϕ}_{n} - 3 {ϕ}_{n - 1} + {ϕ}_{n - 2}}{16 π \cdot γ \cdot {B}_{0} \cdot {G}_{A} \cdot {G}_{D} \cdot {S}_{n}} = \frac{{D}_{n + 1} + 2 {D}_{n} + {D}_{n - 1}}{2} \qquad ( 5 )$$

Here the temperature correction 32 uses the combination comprising the sum Dₙ₊₁+2 Dₙ+Dₙ₋₁ where Dₙ is the computed displacement for the image element at the image frame n, Dₙ₊₁ is the computed displacement for the image element at the succeeding image frame n+1, and Dₙ₋₁ is the computed displacement for the image element at the preceding image frame n−1. The approach of Equation (5) can also be written as

$${D}_{n} + ( \frac{{D}_{n + 1} + {D}_{n - 1}}{2} ) ,$$

so that the temperature correction of computed displacement Dₙ is

$$\frac{{D}_{n + 1} + {D}_{n - 1}}{2} .$$

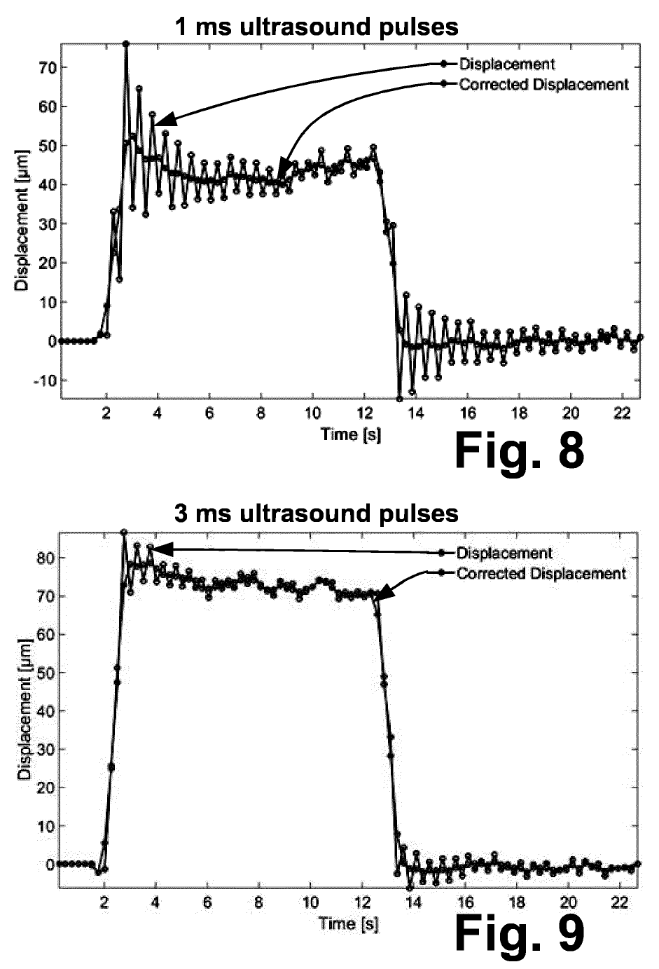

With reference to FIGS. 8 and 9, the displacement Dₙ shown in respective FIGS. 4 and 5 is plotted (“Displacement”) along with the temperature-corrected displacement Dₙ^{C} (“Corrected Displacement”) computed using Equation (4) (or, equivalently, using Equation (5)). It is seen in FIGS. 8 and 9 that the temperature correction provides significant improvement in the stability of the measured displacement.

With reference back to FIGS. 4 and 5, the temperature increase (especially the example of FIG. 4 with lower heating) is seen to be subject to fluctuation at the beginning and the end of the sonication time interval (times 2 sec and 13 sec, respectively, for this example) when the ultrasonic excitation power is turned on or off and rapid variation of the displacement are occurring. The temperature extraction of Equation (3) assumes that the phase variation due to displacement in succeeding dynamics n and n−1 cancel each other out. This assumption is seen in FIGS. 4 and 5 to lose validity at the sonication time interval start and stop times. Without being limited to any particular theory of operation, this is believed to be due to the rapid variation in displacement during these transients.

Since the temperature correction uses the estimation of the temperature Tₙ^{A} (or at least the temperature change estimated therefrom), the temperature correction of the displacement can be improved by improving the estimation of the temperature Tₙ^{A}. In particular, as seen in FIGS. 4 and 5 (especially FIG. 4), the estimated temperature is subject to fluctuation at the beginning and the end of the sonication time interval when the acoustic power was turned on or off. These start and stop times when the acoustic power changes are known since they are controlled by the ultrasound device 12, and so the start and stop times can be inputs to the MR controller 20 of FIG. 1. In some embodiments, these temperature transients are smoothed by interpolation. In a suitable approach, the corresponding dynamic and the following dynamic (because Tₙ^{A} is based on the average of two phase images) can be replaced by an interpolation of dynamics acquired before and after this variation of power.

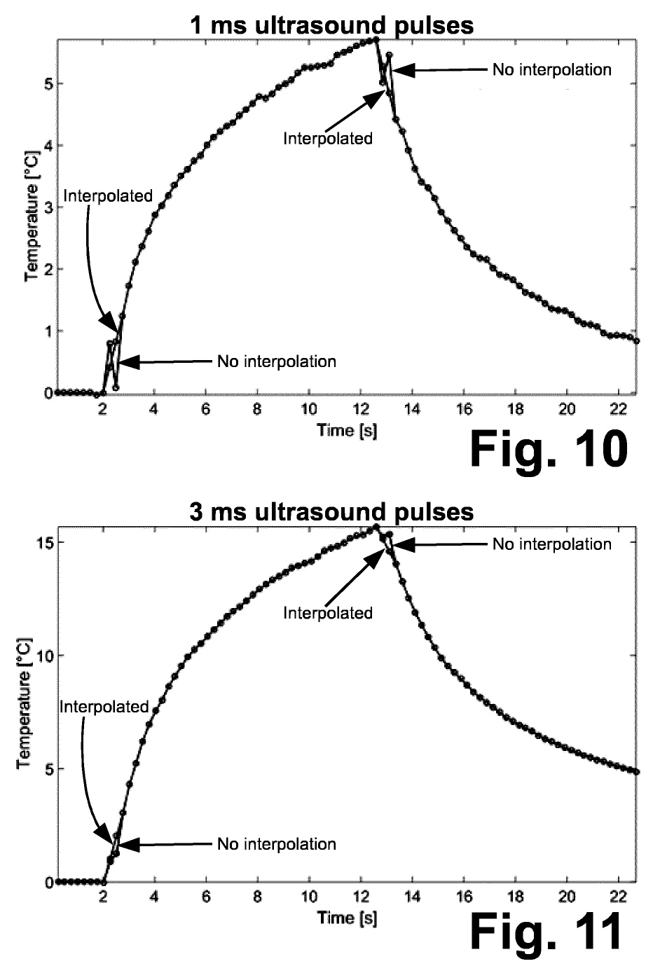

With reference to FIGS. 10 and 11, the resulting interpolated temperature, denoted herein as Tₙ^{I}, is presented for the data of FIGS. 2 and 3, respectively. The corrected displacement Dₙ^{C} can be processed using the corrected interpolated temperature Tₙ^{I} as follows:

$${D}_{n}^{C} = {D}_{n} - \frac{α \cdot {T}_{E}}{2 \cdot {G}_{A} \cdot {G}_{D} \cdot {S}_{n}} \cdot \frac{{T}_{n + 1}^{I} - {T}_{n - 1}^{I}}{2} \qquad ( 6 )$$

which is seen to be equivalent to Equation (4) except for the substitution of interpolated temperature T^{I} for the temperature T^{A} of Equation (3).

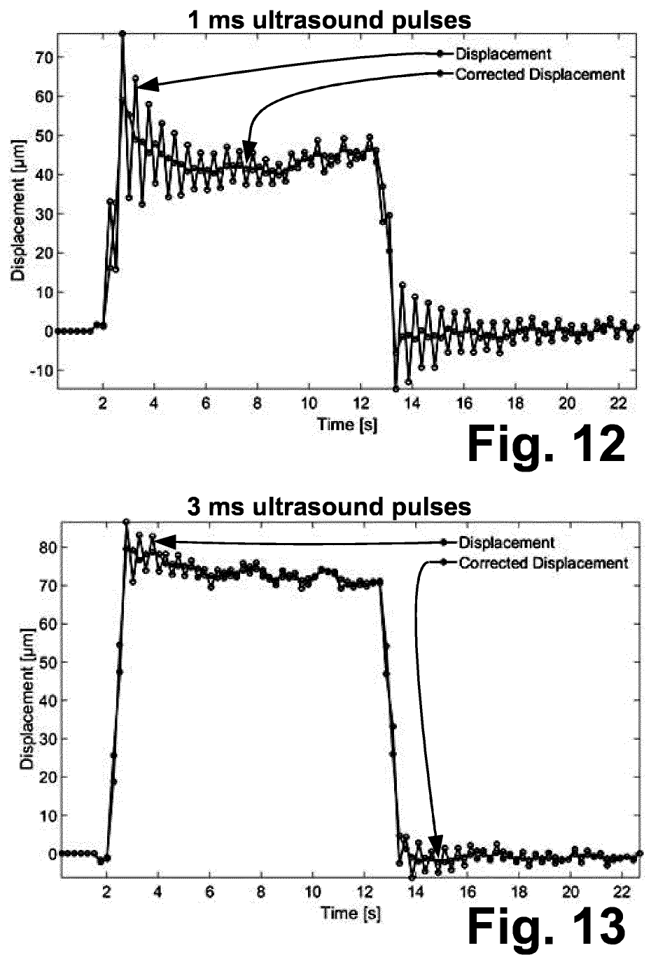

With reference to FIGS. 12 and 13, the displacement Dₙ shown in respective FIGS. 4 and 5 is again plotted (“Displacement”) along with the temperature-corrected displacement Dₙ^{C} (“Corrected Displacement”) computed using Equation (6). Compared to FIGS. 8 and 9, corresponding respective FIGS. 12 and 13 show that using of the interpolated temperature Tₙ^{I} provides a better estimation of the displacement during transition periods of the acoustic power level.

The temperature corrections of the displacement previously described with reference to Equations (4)-(6) require knowledge of the phase φₙ₊₁ and φₙ₊₂ to perform temperature correction for the displacement Dₙ of image frame n, so that these corrections are suitable for post processing, or require a delay (i.e. latency) between image frame acquisition and the temperature correction.

To implement equivalent real-time correction without such a latency, the temperature change at dynamic frame n can be approximated assuming it is close to the temperature variation previously observed, e.g. at image frame n−1. The resulting correction of the displacement can be expressed as:

$${D}_{n}^{C} = {D}_{n} - \frac{α \cdot {T}_{E}}{2 \cdot {G}_{A} \cdot {G}_{D} \cdot {S}_{n}} \cdot ( {T}_{n}^{A} - {T}_{n - 1}^{A} ) \qquad ( 7 )$$

Comparing with Equation (4), it is apparent that the temperature derivative estimation

$$\frac{1}{2} ( {T}_{n + 1}^{A} - {T}_{n - 1}^{A} )$$

of Equation (3D) is replaced by the temperature derivative estimation (Tₙ^{A}−Tₙ₋₁^{A}) which can advantageously be computed in real-time.

In analogy to Equation (5), the corrected displacement D^{C}ₙ of Equation (7) can also be expressed as function of the phase:

$${D}_{n}^{C} = \frac{{ϕ}_{n} - 2 {ϕ}_{n - 1} + {ϕ}_{n - 2}}{8 π \cdot γ \cdot {B}_{0} \cdot {G}_{A} \cdot {G}_{D} \cdot {S}_{n}} = \frac{{D}_{n} + {D}_{n - 1}}{2} \qquad ( 8 )$$

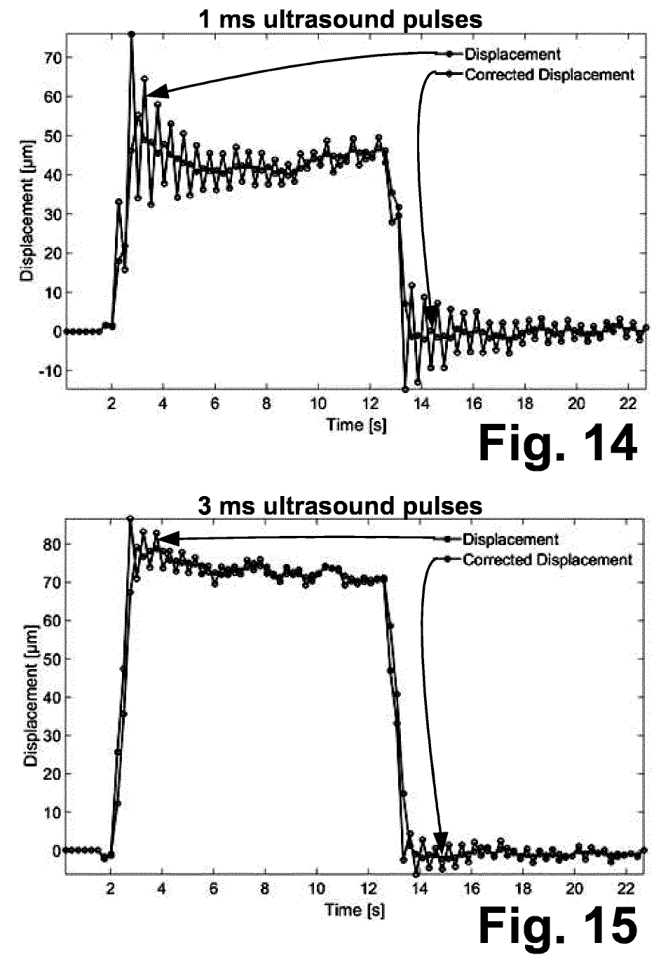

With reference to FIGS. 14 and 15, the displacement Dₙ shown in respective FIGS. 4 and 5 is again plotted (“Displacement”) along with the temperature-corrected displacement Dₙ^{C} (“Corrected Displacement”) computed using Equation (7). Compared to FIGS. 8 and 9, corresponding respective FIGS. 14 and 15 demonstrate that temperature derivative estimation (Tₙ^{A}−Tₙ₋₁^{A}) performs almost as well as other temperature correction approaches. However, it induces a processing latency of half of the dynamic duration in the estimation of the displacement.

As another illustrative embodiment, the temperature derivative estimation used in the temperature correction operation 32 could be (Tₙ₊₁^{A}−Tₙ^{A}). However, this choice of temperature derivative estimation is generally less accurate than that of Equation (3D) and also does not provide the benefit of facilitating real-time processing.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof

1. A Magnetic Resonance Acoustic Radiation Force Imaging (MR-ARFI) apparatus comprising:
a magnetic resonance (MR) imaging device configured to perform gradient echo (GRE) imaging including successive MR dynamics with opposite encoding of displacement to generate MR-ARFI data of a subject in which the MR-AFRI data comprises successive image frames with opposite encoding of displacement;
an ultrasound device configured to apply sonication to the subject over sonication time intervals during the GRE imaging; and
an electronic processor programmed to perform an MR-ARFI data processing method applied to image elements at image frames of the MR-AFRI data including:
computing a displacement for the image element at the image frame as proportional to a difference between the phase of the image element in the image frame and the phase of the image element in a succeeding or preceding image frame with opposite encoding of displacement, and
correcting the computed displacement for a temperature change for the image element between the image frame and the succeeding or preceding image frame to generate a temperature-corrected displacement for the image element at the image frame, wherein the temperature change is determined using the MR-AFRI data.

2. The MR_ARFI apparatus of claim 1 wherein computing the correcting includes:
numerically estimating a temperature derivative for the image element at the image frame from the MR-AFRI data; and
correcting the computed displacement using the temperature derivative to generate the temperature-corrected displacement for the image element at the image frame.

3. The MR-ARFI apparatus of claim 2 wherein the correcting further includes:
generating a temperature versus image frame curve for the image element from the MR-AFRI data; and
performing the numeric estimation of the temperature derivative for the image element at the image frame using the temperature versus image frame curve.

4. The MR-ARFI apparatus of claim 3 wherein the correcting further includes:
at start and stop times of the sonication time intervals, smoothing the temperature versus image frame curve using interpolation;
wherein the smoothed temperature versus image frame curve is used in the numeric estimation of the temperature derivative for the image element at the image frame.

5. The MR_ARFI apparatus of claim 1 wherein correcting includes:
correcting the computed displacement for the temperature change between the image frame and the succeeding or preceding image frame using a combination of at least two of (I) the computed displacement for the image element at the image frame, (II) the computed displacement for the image element at the succeeding image frame, and (III) the computed displacement for the image element at the preceding image frame.

6. The MR_ARFI apparatus of claim 5 wherein the correcting uses the combination comprising the sum Dₙ₊₁2 Dₙ+Dₙ₋₁ where Dₙ is the computed displacement for the image element at the image frame, Dₙ₊₁ is the computed displacement for the image element at the succeeding image frame, and Dₙ₋₁ is the computed displacement for the image element at the preceding image frame.

7. The MR_ARFI apparatus of claim 1 wherein the MR-AFRI data processing method further comprises generating a temperature-corrected displacement image for a displayed image frame comprising the temperature-corrected displacements for the image elements at the displayed image frame.

8. The MR_ARFI apparatus of claim 1 wherein the MR-AFRI data processing method further comprises generating a temperature-corrected displacement versus time profile for a displayed image element comprising the temperature-corrected displacements for the displayed image element plotted against image frame.

9. The MR_ARFI apparatus of claim 7 further comprising:
a display operated by the electronic processor to display the temperature-corrected displacement image or the temperature-corrected displacement versus time profile.

10. A non-transitory storage medium storing instructions readable and executable by an electronic processor to perform a Magnetic Resonance Acoustic Radiation Force Imaging (MR-ARFI) method operating on MR-AFRI data of a subject comprising successive image frames with opposite encoding of displacement acquired during sonication of the subject over sonication time intervals, the MR-ARFI method comprising:
computing a temperature-corrected displacement for an image element at an image frame of the MR-AFRI data from the phase of the image element at the image frame and the phase of the image element at a succeeding or preceding image frame with opposite encoding of displacement; and
repeating the computing for at least one of:
all image elements of the image frame to generate a temperature-corrected displacement image; and
a contiguous plurality of image frames of the MR-AFRI data to generate a temperature-corrected displacement versus time profile for the image element.

11. The non-transitory storage medium of claim 10 wherein computing the temperature-corrected displacement includes:
computing displacement for the image element at the image frame as proportional to a difference between the phase of the image element at the image frame and the phase of the image element at a succeeding or preceding image frame with opposite encoding of displacement;
numerically estimating a temperature derivative for the image element at the image frame from the MR-AFRI data; and
correcting the computed displacement for the image element at the image frame using the temperature derivative to generate the temperature-corrected displacement for the image element at the image frame.

12. The non-transitory storage medium of claim 10 wherein computing the temperature-corrected displacement includes:
computing displacement for the image element at each of the image frame and at least one preceding or succeeding image frame wherein each displacement is computed as proportional to a phase difference for the image element in adjacent image frames with opposite encoding of displacement; and
performing temperature correction using a combination of at least two of (i) the computed displacement for the image element at the image frame, (ii) the computed displacement for the image element at the succeeding image frame, and (iii) the computed displacement for the image element at the preceding image frame.

13. The non-transitory storage medium of claim 12 wherein the temperature correction uses the sum Dₙ₊₁2 Dₙ+Dₙ₋₁ where Dₙ is the computed displacement for the image element at the image frame, Dₙ₊₁ is the computed displacement for the image element at the succeeding image frame, and Dₙ₋₁ is the computed displacement for the image element at the preceding image frame.

14. The non-transitory storage medium of claim 10 wherein the repeating includes:
repeating the computing for all image elements of the image frame to generate the temperature-corrected displacement image.

15. The non-transitory storage medium of claim 10 wherein the repeating includes:
repeating the computing for said contiguous plurality of image frames of the MR-AFRI data to generate the temperature-corrected displacement versus time profile for the image element.

16. The non-transitory storage medium of claim 10 wherein the MR-ARFI method further comprises:
operating a display to display at least one of the temperature-corrected displacement image and the temperature-corrected displacement versus time profile.

17. A Magnetic Resonance Acoustic Radiation Force Imaging (MR-ARFI) method comprising:
performing gradient echo (GRE) imaging using a magnetic resonance (MR) imaging device to acquire MR-ARFI data of a subject in which the MR-AFRI data comprises successive image frames with opposite encoding of displacement;
applying sonication to the subject over sonication time intervals during the GRE imaging using an ultrasound device; and
using an electronic processor, for image elements at image frames of the MR-ARFI data:
(i) computing displacement as proportional to a difference between the phase in the image frame and the phase in a succeeding or preceding image frame with opposite encoding of displacement, and
(ii) correcting the computed displacements for a temperature change between the image frame and the succeeding or preceding image frame wherein the temperature change is determined using the MR-AFRI data.

18. The MR_ARFI method of claim 17 wherein the correcting includes:
numerically estimating a temperature derivative from the MR-AFRI data; and
correcting the computed displacement using the temperature derivative.

19. The MR-ARFI method of claim 18 further comprising:
at start and stop times of the sonication time intervals, smoothing a temperature versus image frame curve derived from the MR-AFRI data and used in the numeric estimation of the temperature derivative.

20. The MR_ARFI method of claim 17 wherein the correcting includes:
correcting for the temperature change using a combination of at least two of (I) the computed displacement of the image frame, (II) the computed displacement of the succeeding image frame, and (III) the computed displacement of the preceding image frame.

21. The MR_ARFI method of claim 20 wherein the correcting uses the combination comprising the sum Dₙ₊₁2 Dₙ+Dₙ₋₁ where Dₙ is the computed displacement of the image frame, Dₙ₊₁ is the computed displacement of the succeeding image frame, and Dₙ₋₁ is the computed displacement of the preceding image frame.
